(19) United States
(12) Patent Application Publication
(10) Pub. No.: US 2026/0345949 A1
(43) Pub. Date: Nov. 5, 2026

(54) Method for delivering a personalized emotional and mental healing experience

(71) Applicant: 9D Breathwork Management Services LLC, Sheridan, WY (US)

(72) Inventor: Brian Patrick Kelly, Petaluma, CA (US)

(73) Assignee: 9D Breathwork Management Services LLC, Sheridan, WY (US)

(21) Appl. No.: 19/460,643

(22) Filed: Jan. 27, 2026

Related U.S. Application Data
(60) Provisional application No. 63/752,123, filed on Jan. 31, 2025.

(51) Int. Cl. A61M 21/02; A61M 21/00

(52) U.S. Cl. CPC A61M 21/02; A61M 2021/0027; A61M 2021/005

(57) ABSTRACT

The present invention provides a method for delivering a personalized emotional and mental healing experience. The method begins with selecting a customized journey based on the user’s specific emotional or mental struggles, such as stress relief, trauma healing, or self-confidence. The experience is enhanced with a multi-dimensional soundscape delivered via 9D stereo audio, incorporating binaural beats and isochronic tones to guide the user’s brain into desired states of relaxation, focus, or energy release. The sound environment is tuned to 432Hz to promote healing and relaxation. Subliminal messaging and hypnotic suggestions are integrated to address limiting beliefs and emotional blockages. The method also includes guided visualizations and immersive sensory experiences, which evolve based on the user’s emotional state. Additionally, bioacoustic sound effects synchronize with the user’s physiological responses to enhance emotional regulation and deepen the mind-body connection. This method supports deep mental and emotional healing.

### CROSS-REFERENCE TO RELATED APPLICATIONS

This non-provisional application claims the benefit of U.S. Provisional Patent Application No. 63/752,123 filed on the Jan. 31^{st}, 2025, the entire contents of which are incorporated herein by reference.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The present invention falls within the field of methods and systems for therapeutic treatment.

There are existing patent documents related to technology-enhanced therapeutic treatments. For example, U.S. Patent 9,079,030 by Holloway reveals a protocol or procedure for lowering sympathetic nervous system arousal in a person with the aim to prepare that person for a medical or dental procedure. To achieve that goal, various steps are undertaken, including, but not limited to, administering a therapeutic dosage of one or more neurotransmitter supplements, placing gelled electrodes adjacent or below the mastoid and connecting them to a cranial electrotherapy stimulation device that administers a sub-sensation level current to a human being. US Patent 7,346,395 by Lozano and Mayberg provides for a method of treating, using neuromodulation, depression, mood disorders, and anxiety disorders. More particularly, the invention disclosed by Lozano and Mayberg involves electrical stimulation and/or chemical stimulation to treat depression. It includes surgically implanting an electrical stimulation lead and/or catheter which is in communication with a predetermined site. The aforementioned site is coupled to a signal generator and/or infusion pump which releases either an electrical signal and/or a pharmaceutical resulting in stimulation of the predetermined site. In this way, the invention by Lozano and Mayberg claims to be capable of treating the anxiety or the mood of an individual. It is also worth mentioning the US Patent Application No. 2006/0136009 by Staffel and Holloway which describes a protocol or procedure for lowing sympathetic nervous system arousal with the aim to prepare a person for a dental or medical procedure. It covers the administration of a therapeutic dosage of one or more neurotransmitter supplements. Afterwards, gelled electrodes are connected to a cranial electrotherapy stimulation device which administers a sub-sensation level current to the patient. Furthermore, the invention provides for placing a noise dampening headset on the patient as well as for playing a neuroacoustic entrainment recording or program. Another patent document that deserves to be mentioned is US Patent Application No. 2007/0203432 by McNew for an apparatus, system, and method for creating an individually balanceable environment of sound and light. The invention disclosed by McNew comprises the steps of exposing a user to spectral light and laterally directed sound. The invention further adjusts the manner of exposure in such a way as to be sufficient to elicit a desired autonomic nervous response.

The existing prior art, including the aforementioned patents, does not disclose any invention that includes breathwork and different sound layers. Breathwork, which broadly can be defined as a practice involving conscious control of breathing patterns, may offer various advantages for healing on physical, mental, and emotional levels. For example, such advantages may include stress reduction, improved oxygenation, detoxification, pain relief, and enhanced immune function. Sound healing, in turn, relies on various sound layers and frequencies to benefit the mind and the body of individuals. In the light of the aforementioned observations, there is a pressing social need for an invention for delivering a personalized emotional and mental healing experience which includes and improves elements of the prior art, but also adds new elements, such as breathwork and different sound layers.

### SUMMARY OF THE INVENTION

The invention is designed to deeply transform the mind, body, and subconscious through a multi-dimensional blend of breathwork, sound frequencies, guided imagery, and personalized therapeutic techniques, all tailored to facilitate emotional healing, mental clarity, and profound relaxation. The invention integrates a combination of binaural beats, isochronic tones, harmonic layers, and 432Hz tuning to guide users into specific brainwave states. This immersive soundscape enhances emotional healing and mental clarity by facilitating deep relaxation, focus, or energy release, depending on the user’s needs. The invention further allows for the customization of breathwork techniques (such as euphoric breathing, holotropic breathing, or deep belly breathing) based on user preference or emotional state. This personalized approach ensures that each session targets the individual's specific mental and emotional needs, maximizing effectiveness. The invention layers subliminal messaging and hypnotic suggestions into the sound experience, thus facilitating subconscious reprogramming. This allows for deep, transformational work, such as overcoming limiting beliefs, healing trauma, and building self-confidence, without the need for conscious effort. The invention also offers a library of sessions, each designed around specific themes to help individuals address the root causes of their struggles. These sessions combine guided visualizations and immersive soundscapes, tailored to evolve in real-time based on the user’s breath and emotional state. The integration of visual imagery with dynamic auditory stimuli creates a deeply immersive experience, allowing users to explore and heal the underlying issues that affect their mental and emotional well-being.

The key features of the invention are described below.

The user selects a journey based on their specific emotional or mental struggles. Each journey is designed to address a particular theme, such as stress relief, trauma healing, or self-confidence, ensuring that the experience is tailored to the user’s current needs.

The system employs a multi-dimensional soundscape delivered through 9D stereo audio, immersing the user in a rich auditory environment that enhances the breathwork experience. This soundscape supports emotional healing, mental clarity, and relaxation by influencing the user’s brainwave patterns.

The session integrates binaural beats and isochronic tones, which are carefully designed to guide the user’s brain into specific states of relaxation, focus, or energy release. These sound frequencies help facilitate deep mental and emotional healing.

432Hz tuning is used throughout the experience to promote healing and harmony. This frequency is known for its ability to resonate with the body and mind on a deep level, fostering relaxation, reducing stress, and encouraging a sense of well-being.

Subliminal messaging and hypnotic suggestions are seamlessly layered into the sound experience, facilitating subconscious reprogramming. This layer addresses underlying issues such as limiting beliefs, past trauma, and emotional blockages, supporting deep transformational healing.

Guided visualizations are paired with immersive sensory experiences, including dynamic soundscapes that evolve based on the user’s emotional state. This combination of auditory and visual stimuli enhances the healing journey, helping the user address and process emotional challenges.

Bioacoustic sound effects are used to synchronize with the user’s physiological responses, such as heart rate or breath patterns, further enhancing the healing process. These sounds support emotional regulation and deepen the connection between the body and mind during the session.

The invention provides a holistic and immersive healing experience tailored to individual needs. Furthermore, it combines advanced audio technologies, guided therapy techniques, and physiological feedback for maximum efficacy. The invention also constitutes a non-invasive and accessible solution for emotional and mental health challenges.

The invention uniquely integrates a combination of personalized breathwork techniques, multi-dimensional sound therapy (including binaural beats, isochronic tones, harmonic layers, and 432Hz tuning), subliminal messaging, hypnotic therapy, and guided visualizations. This innovative fusion of therapeutic methods allows for a fully immersive and adaptive experience, targeting emotional healing, mental clarity, and relaxation in a way that is personalized to the individual’s specific needs. The invention’s library of sessions, each centered around specific therapeutic themes (e.g., trauma healing, stress relief, self-confidence), is a unique feature. The personalized and interactive nature of these sessions, along with their ability to help users dive deeper into their healing process, distinguishes the system as an innovative tool for emotional and psychological transformation. The invention provides a unique, accessible system that integrates multiple healing techniques—breathwork, sound therapy, subconscious reprogramming, and immersive visual experiences—to offer a comprehensive method for stress relief, emotional healing, and mental well-being. The ease of use combined with the profound therapeutic impact positions the invention as a novel and non-obvious solution in the wellness and mental health space.

The innovation centers on a comprehensive, multi-dimensional approach to emotional healing and mental clarity. By integrating personalized breathwork techniques, immersive sound therapy, subconscious reprogramming, and guided visualizations, the system offers a deeply transformative experience. The innovation lies in how these diverse therapeutic elements are combined into a cohesive, adaptive system that is tailored to the individual’s specific emotional and physiological needs, making it an effective tool for personal growth, mental well-being, and stress relief across various settings.

This invention can be applied in mental health therapies, wellness programs, mindfulness practices, stress management, trauma recovery, and personal development initiatives.

### BRIEF DESCRIPTION OF THE DRAWINGS

FIG. 1, according to an embodiment of the present invention, is an illustration of a flow-chart that explains the method for delivering a personalized emotional and mental healing experience.

### FIGURES– REFERENCE NUMERALS

10 – Start of the process for delivering a personalized emotional and mental healing experience
20 – Selecting a journey
30 – Delivering a soundscape
40 – Integrating beats and tones
50 – Tuning the sound experience
60 – Embedding subliminal messaging and hypnotic suggestions
70 – Incorporating guided visualizations
80 – Applying bioacoustics sound effects
90 – End of the process for delivering a personalized emotional and mental healing experience

### DETAILED DESCRIPTION OF THE INVENTION

In the following detailed description, a reference is made to the accompanying drawing that forms a part hereof, and in which the specific embodiments that may be practiced are shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

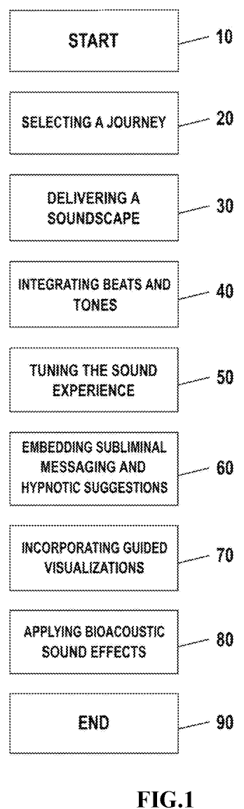

Referring now to the invention in more detail, in FIG. 1, there is shown an illustration of a flow-chart that explains the method for delivering a personalized emotional and mental healing experience. The process described in FIG. 1 consists of nine steps, namely: start of the process for delivering a personalized emotional and mental healing experience 10; selecting a journey 20; delivering a soundscape 30; integrating beats and tones 40; tuning the sound experience 50; embedding subliminal messaging and hypnotic suggestions 60; incorporating guided visualizations 70; applying bioacoustics sound effects 80; and end of the process for delivering a personalized emotional and mental healing experience 90.

In more detail, still referring to the invention of FIG. 1, after the start of the method for delivering a personalized emotional and mental healing experience 10, the user of the invention proceeds with selecting a journey 20. The journey is based on the specific emotional or mental struggles of the user. The journey is designed to address a particular theme, such as stress relief, trauma healing, or self-confidence, ensuring that the experience is tailored to the user’s current needs. After selecting a journey 20, the invention proceeds with delivering a soundscape 30. More particularly, a multi-dimensional soundscape is delivered through 9D stereo audio, immersing the user in a rich auditory environment that enhances the breathwork experience. This soundscape supports emotional healing, mental clarity, and relaxation by influencing the user’s brainwave patterns. After delivering a soundscape 30, the invention proceeds with integrating beats and tones 40. The binaural beats and isochronic tones are carefully designed to guide the user’s brain into specific states of relaxation, focus, or energy release. These sound frequencies help facilitate deep mental and emotional healing. After integrating beats and tones 40, the invention proceeds with tuning the sound experience 50. 432Hz tuning is used throughout the experience to promote healing and harmony. This frequency is known for its ability to resonate with the body and mind on a deep level, fostering relaxation, reducing stress, and encouraging a sense of well-being. After tuning the sound experience 50, the invention proceeds with embedding subliminal messaging and hypnotic suggestions 60. Subliminal messaging and hypnotic suggestions are seamlessly layered into the sound experience, thus facilitating subconscious reprogramming. This step addresses underlying issues such as limiting beliefs, past trauma, and emotional blockages, supporting deep transformational healing. After embedding subliminal messaging and hypnotic suggestions 60, the invention proceeds with incorporating guided visualizations 70. Guided visualizations are paired with immersive sensory experiences, including dynamic soundscapes that evolve based on the user’s emotional state. This combination of auditory and visual stimuli enhances the healing journey, helping the user address and process emotional challenges. After incorporating guided visualizations 70, the next step is applying bioacoustics sound effects 80. Bioacoustic sound effects are used to synchronize with the user’s physiological responses, such as heart rate or breath patterns, further enhancing the healing process. These sounds support emotional regulation and deepen the connection between the body and mind during the session. After applying bioacoustics sound effects 80, the next step is the end of the method for delivering a personalized emotional and mental healing experience 90.

Regarding selecting a journey 20, the user selects a journey from a plurality of available themes, including but not limited to leadership, grief and loss, abundance, stress and anxiety, depression, and interpersonal relationships. As for delivering a soundscape 30, the system provides access to a library of soundscapes comprising a plurality of music genres and associated emotional progressions, enabling customization of the user experience. In relation to integrating beats and tones 40, user can select desired brain entrainment frequencies according to their preference and to what brain state/s they want to stimulate (Delta 0.5-4 Hz, Theta (4-8 Hz), Alpha (8-12 Hz), Beta (12-30 Hz), Gamma (30-100+ Hz). They can also select among binaural and monaural frequencies, isochronic tones, or a blend of them. Pertaining to tuning the sound experience 50, this may include final touches like selecting the vocal guide (male/female/custom voices), breathing pattern model, volume balancing (between soundscape, vocal guide, binaural beats). In relation to embedding subliminal messaging and hypnotic suggestions 60, such messaging can be tailored to the selected narrative / theme of the experience. “I am a true leader”, “I am confident about each step I give”, “I was born to lead” could be subliminal messages for an “Unleash your Leadership” session. With regard to applying bioacoustics sound effects 80, such effects may include, but are not limited to, rain, river/sea/ocean sound, forest ambiences. Concerning the end of the process for delivering a personalized emotional and mental healing experience 90, the tailored session is now ready to be played in a friendly and simple digital interface that allows to play/stop, forward/rewind, play offline, etc.

The present invention as shown in FIG. 1 may be implemented by hardware, software, or a combination of both. The present invention may be implemented through codes or instructions in a software application that can be implemented by a processor. Such a processor may include, for instance, microprocessor in a mobile terminal.

The embodiment is able to be implemented, for example, using a machine-readable medium or article which is able to store an instruction or a set of instructions that, if executed by a machine, cause the machine to perform the operations described herein. Such machine is able to include, for example, any suitable processing platform, computing platform, computing device, processing device, electronic device, electronic system, computing system, processing system, computer, processor, or the like, and is able to be implemented using any suitable combination of hardware and/or software.

The machine-readable medium or article is able to include, for example, any suitable type of memory unit, memory device, memory article, memory medium, storage device, storage article, storage medium and/or storage unit; for example, memory, removable or non-removable media, erasable or non-erasable media, write-able or re-write-able media, digital or analog media, hard disk drive, floppy disk, Compact Disk Read Only Memory (CD-ROM), Compact Disk Recordable (CD-R), Compact Disk Re-Writeable (CD-RW), optical disk, magnetic media, various types of Digital Versatile Disks (DVDs), a tape, a cassette, or the like.

The instructions are able to include any suitable type of code, for example, source code, compiled code, interpreted code, executable code, static code, dynamic code, or the like, and are able to be implemented using any suitable high-level, low-level, object-oriented, visual, compiled and/or interpreted programming language, e.g., C, C++, Java, BASIC, Pascal, Fortran, Cobol, assembly language, machine code, or the like. Functions, operations, components and/or features described herein with reference to one or more embodiments, are able to be combined with, or are able to be utilized in combination with, one or more other functions, operations, components and/or features described herein with reference to one or more other embodiments, or vice versa.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

I claim:

1. A computer-implemented method for providing a personalized emotional and mental healing experience, comprising the steps of:
selecting a journey customized to the user’s specific emotional or mental struggles, where the journey is tailored to address a particular theme, such as stress relief, trauma healing, and self-confidence;
delivering a multi-dimensional soundscape via 9D stereo audio to immerse the user in an auditory environment that enhances a breathwork experience and influences the user’s brainwave patterns for emotional healing, mental clarity, and relaxation;
integrating binaural beats and isochronic tones within the soundscape, the frequencies of which are designed to guide the user’s brain into specific states of relaxation, focus, or energy release;
tuning the sound experience to a 432Hz frequency, to promote healing, harmony, and relaxation in the user;
embedding subliminal messaging and hypnotic suggestions into the sound experience to facilitate subconscious reprogramming and address issues such as limiting beliefs, past trauma, and emotional blockages;
incorporating guided visualizations paired with immersive sensory experiences that evolve dynamically based on the user’s emotional state, enhancing the healing process through both auditory and visual stimuli;
applying bioacoustic sound effects that synchronize with the user’s physiological responses, such as heart rate or breath patterns, to further enhance emotional regulation and deepen the connection between the body and mind.

2. The method of claim 1, wherein, to ensure that each breathwork journey is perfectly aligned with the user’s current state and to promote the maximum relaxation or emotional release, the method uses real-time biofeedback, such as heart rate or breath patterns, to adapt the session, adjusting the sound frequencies, breathing cues, and session intensity according to the user's emotional and physiological responses.

3. The method of claim 1, wherein the method features AI-driven coaching that provides real-time feedback and guidance during sessions and this AI-driven coaching helps users stay engaged, offers personalized encouragement, and adjusts instructions to match the users’ progress, thus ensuring a deeper connection to the breathwork practice.

4. The method of claim 1, wherein the steps presented in claim 1 can have a different order.

5. The method of claim 1, wherein only two or more, but less than all, of the steps presented in claim 1 are actually used by the user.

6. The method of claim 1, wherein only two or more, but less than all, of the steps presented in claim 1 are actually used by the user and wherein the used steps can have a different order than those presented in claim 1.

7. The method of claim 1, wherein only two or more, but less than all, of the steps presented in claim 1 are actually used by the user, wherein the used steps can have a different order than those presented in claim 1, and, wherein, to ensure that each breathwork journey is perfectly aligned with the user’s current state and to promote the maximum relaxation or emotional release, the method uses real-time biofeedback, such as heart rate or breath patterns, to adapt the session, adjusting the sound frequencies, breathing cues, and session intensity according to the user’s emotional and physiological responses.

8. The method of claim 1, wherein only two or more, but less than all, of the steps presented in claim 1 are actually used by the user, wherein the used steps can have a different order than those presented in claim 1, and, wherein the method features AI-driven coaching that provides real-time feedback and guidance during sessions and this AI-driven coaching helps users stay engaged, offers personalized encouragement, and adjusts instructions to match the user’s progress, thus ensuring a deeper connection to the breathwork practice.
